(19) United States
(12) Patent Application Publication
(10) Pub. No.: US 2022/0257542 A1
(43) Pub. Date: Aug. 18, 2022

(54) PHARMACEUTICAL COMPOSITION COMPRISING TRIMETHOBENZAMIDE OR PHARMACEUTICALLY ACCEPTABLE SALT THEREOF AS ACTIVE INGREDIENT FOR PREVENTING OR TREATING NEUROPATHIC PAIN

(71) Applicant: FRONTBIO INC., Chuncheon-si Gangwon-do (KR)

(72) Inventors: Soo Hyun Park, Chuncheon-si, Gangwon-do (KR); Set Byeol Kim, Chuncheon-si, Gangwon-do (KR)

(21) Appl. No.: 17/630,652

(22) PCT Filed: Jul. 30, 2019
(86) PCT No.: PCT/KR2019/009469

§ 371 Date: Jan. 27, 2022

(51) Int. Cl. A61K 31/166; A61K 31/135

(52) U.S. Cl. CPC A61K 31/166; A61K 31/135

(57) ABSTRACT

The present invention relates to a pharmaceutical composition comprising trimethobenzamide or a pharmaceutically acceptable salt thereof as an active ingredient for preventing or treating neuropathy. Trimethobenzamide, when administered in combination with tramadol, exhibits the effect of significantly reducing pain in a neuropathy animal model and as such, can be advantageously used for treatment of neuropathy.

### BACKGROUND OF THE INVENTION

### 1. Field of the Invention

The present invention relates to a pharmaceutical composition comprising trimethobenzamide or a pharmaceutically acceptable salt thereof as an active ingredient for preventing or treating neuropathy.

### 2. Description of the Related Art

Diabetes is a disease with a rapidly increasing incidence worldwide, and the financial and social costs of diabetes are gradually increasing. In addition, the mortality rate due to various complications accompanying diabetes is also increasing. However, strict control of blood sugar can prevent the progression of the disease or slow the progression of the disease, so maintaining a constant blood sugar level through lifestyle changes and appropriate treatment is considered as an important diabetes prevention method.

Diabetic peripheral neuropathy, one of the main causes of neuropathy, is the most common complication of diabetes, and refers to all signs and symptoms of peripheral nerve dysfunction caused by diabetes. This lowers the quality of life of diabetic patients and increases the mortality rate, and its prevalence is increased according to the patient's age and duration of treatment. In particular, diabetic peripheral neuropathy is more common in type 2 diabetic patients (32.1%) than in type 1 diabetic patients (22.7%) (Primary Care Diabetes, Volume 1, Issue 3, September 2007).

About 10% of diabetic patients complain of persistent pain, which can be spontaneous or induced by stimulation, and may be intractable. The pain caused by such diabetic peripheral neuropathy is usually worse at night and is mainly described as burning pain, stinging pain, aching pain, or a feeling of tightness. In addition, such pain is more clearly felt in the foot than in the hand, and sometimes sudden pain causes weight loss or depression (Pain medicine, Volume 8, Number S2, 2007).

Alpha fatty acid, aldose reductase inhibitor, or gamma-linolenic acid is used as therapeutic agents for diabetic neuropathy. Alpha fatty acid, an antioxidant, prevents the disease progression by reducing oxidative stress, and aldose reductase inhibitor blocks the disease progression by inhibiting the accumulation of sorbitol, which damages nerve cells by preventing glucose from entering the polyol pathway in nerve cells. However, development of aldose reductase inhibitor was mostly stopped at the development stage due to the problems such as side effects and lack of effectiveness, and currently only Epalrestat is used in Japan. On the other hand, linolenic acid, a component of phospholipids constituting the nerve membrane, exhibits an effect of ameliorating some symptoms of diabetic neuropathy by maintaining homeostasis of nerve blood flow.

Meanwhile, trimethobenzamide is used for the treatment of vomiting after surgery or caused by gastroenteritis. This transmits the emetic stimulus to the central nervous system so that the chemoreceptors in the medulla oblongata suppress the emetic stimulus, and it acts by antagonizing the dopaminergic and serotonin receptors. In addition, tramadol is a compound used as an analgesic for general pain. In particular, it is used in the form of chlorohydrate. Tramadol exhibits painkilling effects by enhancing the activation of opioid receptors and the concentration of monoaminergic synapses in neurons. In this regard, Korean Patent Publication No. 10-2008-0005429 discloses a combination of tramadol for the treatment of neuropathic pain.

Accordingly, the present inventors have been trying to develop a drug that can be used for the treatment of neuropathy. In the above process, the present inventors completed the present invention by confirming that a significant pain-suppressing effect appeared when trimethobenzamide was treated alone or in combination with tramadol in an animal model of neuropathy.

### SUMMARY OF THE INVENTION

It is an object of the present invention to provide a pharmaceutical composition comprising trimethobenzamide or a pharmaceutically acceptable salt thereof as an active ingredient for preventing or treating neuropathy.

It is another object of the present invention to provide a pharmaceutical kit for preventing or treating neuropathy comprising trimethobenzamide or a pharmaceutically acceptable salt thereof and tramadol or a pharmaceutically acceptable salt thereof.

To achieve the above objects, the present invention provides a pharmaceutical composition comprising trimethobenzamide or pharmaceutically acceptable salt thereof as an active ingredient for preventing or treating neuropathy.

In addition, the present invention provides a pharmaceutical kit for preventing or treating neuropathy comprising a first component containing a pharmaceutically treatable amount of trimethobenzamide W or a pharmaceutically acceptable salt thereof as an active ingredient; and a second component containing tramadol or a pharmaceutically acceptable salt thereof as an active ingredient.

### Advantageous Effect

When the trimethobenzamide of the present invention is administered in combination with tramadol, it exhibits the effect of significantly reducing pain in a neuropathy animal model, and thus can be effectively used in the treatment of neuropathy.

### BRIEF DESCRIPTION OF THE DRAWINGS

FIG. 1 is a graph confirming the pain relief in a neuropathy animal model by trimethobenzamide.

FIG. 2 is a graph comparing and confirming the pain relief in a neuropathy animal model by single and combined administration of trimethobenzamide and tramadol.

FIG. 3 is a graph confirming the concentration-dependent pain relief in a neuropathy animal model by combined administration of trimethobenzamide and tramadol.

### DESCRIPTION OF THE PREFERRED EMBODIMENTS

Hereinafter, the present invention is described in detail.

The present invention provides a pharmaceutical composition comprising trimethobenzamide or a pharmaceutically acceptable salt thereof as an active ingredient for preventing or treating neuropathy.

The said trimethobenzamide can be a compound represented by the following formula 1:

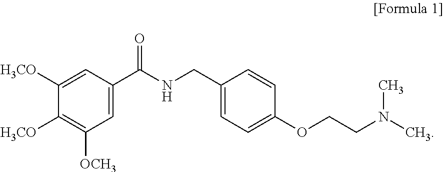 [SMILES: COc1cc(C(=O)NCc2ccc(OCCN(C)C)cc2)cc(OC)c1OC]

The trimethobenzamide can be extracted or synthesized according to the methods known in the art, or purchased and used in a commercially purified state.

The pharmaceutical composition can further include tramadol or a pharmaceutically acceptable salt thereof, or a mixture thereof. The tramadol can be extracted or synthesized according to the methods known in the art, or purchased and used in a commercially purified state.

The said tramadol can be a compound represented by the following formula 2:

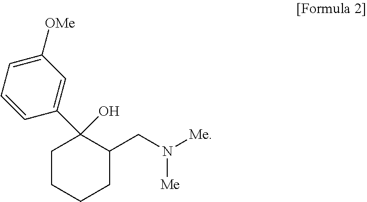 [SMILES: COc1cccc(C2(O)CCCCC2CN(C)C)c1]

The neuropathy can be at least one neuropathy selected from the group consisting of diabetic neuropathy, postherpetic neuropathy, trigeminal neuropathy, peripheral neuropathies, peripheral mononeuropathy, mononeuritis multiplex, autonomic neuropathy, inflammatory neuropathy, toxic or drug-induced neuropathy, nutritional neuropathy, vasculitic neuropathy, paraneoplastic neuropathy, traumatic neuropathy, hereditary neuropathy and idiopathic neuropathy, but not always limited thereto.

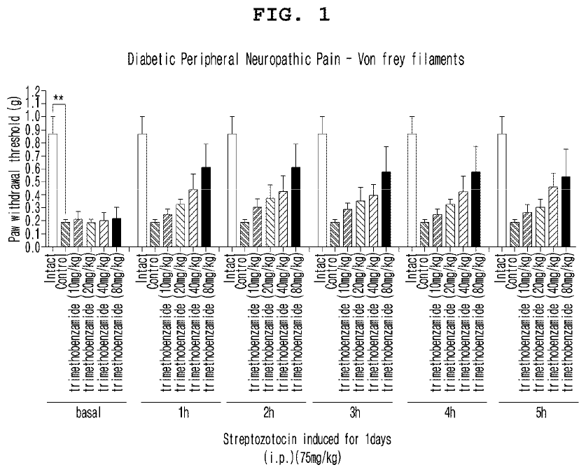

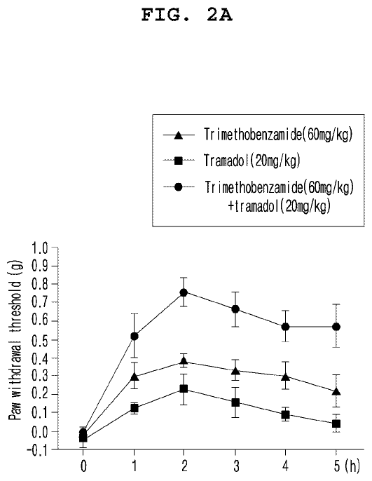

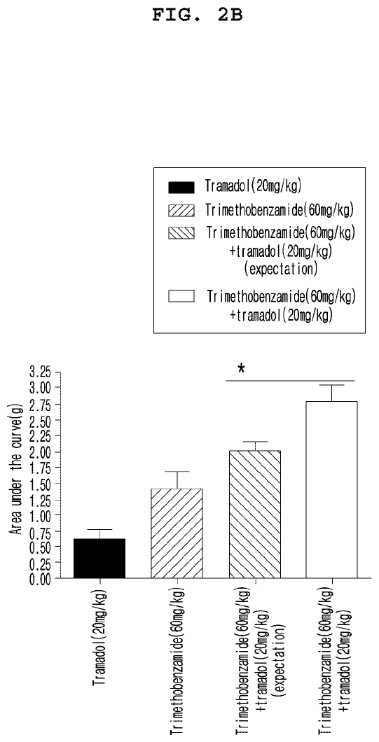

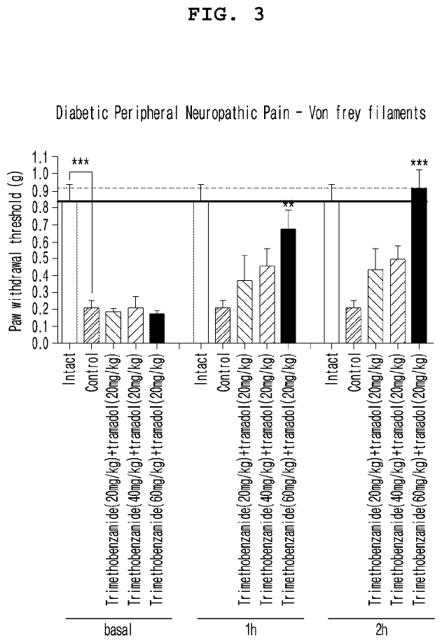

In a preferred embodiment of the present invention, the present inventors confirmed that pain was relieved in a concentration-dependent manner when trimethobenzamide was administered to diabetic neuropathy animal model mice (see FIG. 1). It was also confirmed that the combination of trimethobenzamide and tramadol inhibited neuropathy better than the single administration (see FIGS. 2 and 3).

Therefore, the trimethobenzamide can be used in the treatment of neuropathy by co-administration with tramadol.

The present invention also provides a pharmaceutically acceptable salt of trimethobenzamide or tramadol. The pharmaceutically acceptable salt should have low toxicity to the human body and should not adversely affect the biological activity and physicochemical properties of the compound. The pharmaceutically acceptable salt can include acid addition salts of pharmaceutically usable free acids and basic compounds of trimethobenzamide or tramadol, alkali metal salts (such as sodium salts) and alkali earth metal salts (such as calcium salts), organic base addition salt of carboxylic acid of trimethobenzamide or tramadol with organic base, amino acid addition salt, etc.

The preferred salt forms of the compounds according to the invention include salts with inorganic or organic acids. In this case, the inorganic acid can be hydrochloric acid, sulfuric acid, nitric acid, phosphoric acid, perchloric acid, bromic acid, and the like. In addition, the organic acids include acetic acid, methanesulfonic acid, ethanesulfonic acid, p-toluenesulfonic acid, fumaric acid, maleic acid, malonic acid, phthalic acid, succinic acid, lactic acid, citric acid, gluconic acid, tartaric acid, salicylic acid, malic acid, oxalic acid, benzoic acid, embonic acid, aspartic acid, glutamic acid and the like can be used. Organic bases that can be used in the preparation of the organic base addition salt include tris(hydroxymethyl)methylamine, dicyclohexylamine, and the like. Amino acids that can be used in the preparation of the amino acid addition base include natural amino acids such as alanine and glycine.

Such salts can be prepared by the conventional method. For example, in order to prepare such salts, trimethobenzamide and tramadol or pharmaceutically acceptable salts thereof are dissolved in water-miscible solvents such as methanol, ethanol, acetone and 1,4 dioxane, to which free acids or free bases are added thereto to induce crystallization.

In addition, the compounds according to the present invention can have asymmetric carbon centers and thus may exist as R or S isomers, racemic compounds, individual enantiomers or mixtures thereof, individual diastereomers or mixtures thereof, and these all stereoisomers and mixtures thereof are included within the scope of this invention.

Further, the hydrate or solvate forms of trimethobenzamide and tramadol are also included in the scope of the present invention. Such hydrates or solvates can be prepared by known methods, and it is preferred that they are nontoxic and water-soluble. In particular, it is preferable that these are hydrates or solvates in which 1 to 5 molecules of water or an alcoholic solvent (especially, ethanol, etc.) are bonded.

The pharmaceutical composition according to the present invention can contain trimethobenzamide or a pharmaceutically acceptable salt thereof, an active ingredient, in an amount of 10 to 95 weight % based on the total weight of the composition. In addition, the pharmaceutical composition of the present invention may further include at least one active ingredient exhibiting the same or similar function in addition to the active ingredient.

The pharmaceutical composition of the present invention can contain a generally used carrier, diluent, excipient, or a mixture thereof. The pharmaceutically acceptable carrier can be any carrier that is able to deliver the composition of the present invention in human body without limitation, which is exemplified by the compounds described in Merck Index, 13^{th} ed., Merck & Co. Inc., such as saline, sterilized water, Ringer's solution, dextrose solution, maltodextrin solution, glycerol, ethanol, or a mixture thereof. If necessary, a general additive such as antioxidant, buffer, and bacteriostatic agent can be additionally added.

The composition of the present invention can be formulated by adding with generally used fillers, extenders, binders, wetting agents, disintegrating agents, diluents such as surfactant, or excipients.

The composition of the present invention can be formulated as an oral preparation or a parenteral preparation. The oral preparation may include a solid formulation and a liquid formulation. The solid formulation may be tablets, pills, powders, granules, capsules, or troches, and the solid formulation can be prepared by adding at least one excipient to the composition. The excipient can be starch, calcium carbonate, sucrose, lactose, gelatin, or a mixture thereof. In addition, the solid formulation can include a lubricant, and the lubricant includes magnesium stearate, talc, and the like. Meanwhile, the liquid formulation may be suspensions, solutions, emulsions, or syrups. In this case, the liquid formulation can contain excipients such as wetting agents, sweetening agents, fragrances, and preservatives.

The parenteral preparation can include injections, suppositories, powders for respiratory inhalation, aerosols for spray, powders and creams. The injection may include sterile aqueous solutions, non-aqueous solutions, suspensions, emulsions, and the like. In this case, as the non-aqueous solution or suspension, polyethylene glycol, vegetable oil like olive oil, or injectable ester like ethylolate can be used.

In addition, the present invention provides a pharmaceutical kit for preventing or treating neuropathy comprising a first component containing a pharmaceutically treatable amount of trimethobenzamide or a pharmaceutically acceptable salt thereof as an active ingredient; and a second component containing tramadol or a pharmaceutically acceptable salt thereof as an active ingredient.

The neuropathy can be at least one neuropathy selected from the group consisting of diabetic neuropathy, postherpetic neuropathy, trigeminal neuropathy, peripheral neuropathies, peripheral mononeuropathy, mononeuritis multiplex, autonomic neuropathy, inflammatory neuropathy, toxic or drug-induced neuropathy, nutritional neuropathy, vasculitic neuropathy, paraneoplastic neuropathy, traumatic neuropathy, hereditary neuropathy and idiopathic neuropathy, but not always limited thereto.

Trimethobenzamide or a pharmaceutically acceptable salt thereof, and tramadol or a pharmaceutically acceptable salt thereof included in the pharmaceutical kit may have the characteristics as described above.

For the prevention or treatment of neuropathy, the first and second components of the pharmaceutical kit of the present invention can be administered sequentially or simultaneously. By co-administering the first component and the second component included in the pharmaceutical kit of the present invention, neuropathy can be alleviated.

The first component and the second component included in the kit of the present invention can be administered orally or parenterally according to a desired method. Parenteral administration can include intraperitoneal, intrarectal, subcutaneous, intravenous, intramuscular or intrathoracic injection.

The pharmaceutically treatable amounts of the first component and the second component can be determined according to the type of disease, the severity, the activity of the drug, the patient's sensitivity to the drug, the time of administration, the route of administration, the duration of treatment, the drugs being used simultaneously, and the like. However, for the desired effect, the amount of the first component according to the present invention can be 30 to 90 mg/kg, specifically 40 to 80 mg/kg, and more specifically 50 to 70 mg/kg. In a preferred embodiment of the present invention, the first component can be administered in an amount of 60 mg/kg. Meanwhile, the second component can be administered in an amount of 5 to 35 mg/kg, specifically 10 to 30 mg/kg, and more specifically 15 to 25 mg/kg. In a preferred embodiment of the present invention, the second component can be administered in an amount of 20 mg/kg.

In a preferred embodiment of the present invention, the present inventors confirmed that pain was relieved in a concentration-dependent manner when trimethobenzamide was administered to diabetic neuropathy animal model mice (see FIG. 1). It was also confirmed that the combination of trimethobenzamide and tramadol inhibited neuropathy better than the single administration (see FIGS. 2 and 3).

Therefore, the trimethobenzamide can be used in the treatment of neuropathy by co-administration with tramadol.

Hereinafter, the present invention will be described in detail by the following examples.

However, the following examples are only for illustrating the present invention, and the contents of the present invention are not limited thereto.

### Example 1: Preparation of Neuropathy Animal Model

First, a neuropathy animal model was prepared by administering streptozocin. Specifically, streptozotocin was intraperitoneally administered to 4-week-old ICR mice at the concentration of 75 mg/kg. Five weeks after the administration, the expression of neuropathy was confirmed through the level of blood glucose and Von Frey filament stimulation tests, and the mice were used for the following experiments.

### Experimental Example 1: Confirmation of Inhibition of Neuropathy by Trimethobenzamide

The pain inhibitory effect of the trimethobenzamide compound was confirmed using the animal model prepared in <Example 1>.

Specifically, trimethobenzamide was dissolved in physiological saline to prepare trimethobenzamide solutions with the concentrations of 10, 20, 40 and 80 mg/kg. The prepared trimethobenzamide solutions were orally administered to neuropathy model mice, and Von Frey filament stimulation test was performed 1, 2, 3, 4 and 5 hours after the administration to confirm the inhibitory effect on neuropathy.

As a result, as shown in FIG. 1, it was confirmed that it had a significant effect on the inhibition of neuropathy in a concentration-dependent manner when trimethobenzamide was administered alone (FIG. 1).

### Experimental Example 2: Confirmation of Inhibition of Neuropathy by Co-Administration of Trimethobenzamide

By co-administering trimethobenzamide and tramadol to the animal model prepared in <Example 1>, the pain inhibitory effect was confirmed.

Specifically, trimethobenzamide or tramadol was dissolved in physiological saline to prepare trimethobenzamide solution with the concentration of 60 mg/kg, or tramadol solution with the concentration of 20 mg/kg. After co-administration of the tramadol solution with the concentration of 20 mg/kg and the trimethobenzamide solution with the concentration of 20, 40 or 60 mg/kg solution, the effect of the co-administration was confirmed. The solutions were orally administered to neuropathy model mice, and the Von Frey filament stimulation test was performed 1, 2, 3, 4 and 5 hours after the administration to confirm the inhibitory effect on neuropathy.

As a result, as shown in FIGS. 2 and 3, the pain inhibitory effect was significantly increased when trimethobenzamide and tramadol were co-administered compared to trimethobenzamide or tramadol alone (FIG. 2). In addition, it was confirmed that the pain inhibitory effect was increased as the concentration of trimethobenzamide was increased in the co-administration (FIG. 3).

### INDUSTRIAL APPLICABILITY

When the trimethobenzamide of the present invention is administered in combination with tramadol, it exhibits the effect of significantly reducing pain in a neuropathy animal model, and thus can be effectively used in the treatment of neuropathy.

1. A method of treating neuropathy comprising administering trimethobenzamide or a pharmaceutically acceptable salt thereof to a subject in need thereof.

2. The method of treating neuropathy according to claim 1, wherein the trimethobenzamide is a compound represented by the following formula 1:

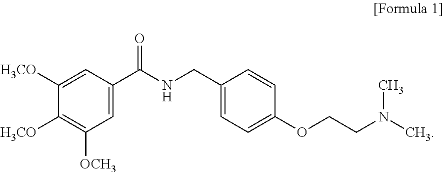 [SMILES: COc1cc(C(=O)NCc2ccc(OCCN(C)C)cc2)cc(OC)c1OC]

3. The method of treating neuropathy according to claim 1, wherein tramadol or a pharmaceutically acceptable salt thereof is further included.

4. The method of treating neuropathy according to claim 3, wherein the tramadol is a compound represented by the following formula 2:

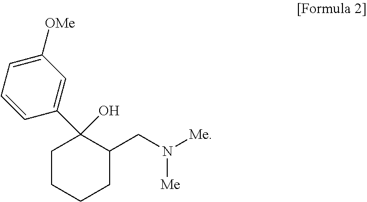 [SMILES: COc1cccc(C2(O)CCCCC2CN(C)C)c1]

5. The method of treating neuropathy according to claim 1, wherein the neuropathy is at least one neuropathy selected from the group consisting of diabetic neuropathy, postherpetic neuropathy, trigeminal neuropathy, peripheral neuropathies, peripheral mononeuropathy, mononeuritis multiplex, autonomic neuropathy, inflammatory neuropathy, toxic or drug-induced neuropathy, nutritional neuropathy, vasculitic neuropathy, paraneoplastic neuropathy, traumatic neuropathy, hereditary neuropathy and idiopathic neuropathy.

6. A pharmaceutical kit for treating neuropathy comprising a first component containing a pharmaceutically treatable amount of trimethobenzamide or a pharmaceutically acceptable salt thereof as an active ingredient; and a second component containing tramadol or a pharmaceutically acceptable salt thereof as an active ingredient.

7. The pharmaceutical kit for treating neuropathy according to claim 6, wherein the first component and the second component are administered sequentially or simultaneously.

8. The pharmaceutical kit for treating neuropathy according to claim 6, wherein the neuropathy is at least one neuropathy selected from the group consisting of diabetic neuropathy, postherpetic neuropathy, trigeminal neuropathy, peripheral neuropathies, peripheral mononeuropathy, mononeuritis multiplex, autonomic neuropathy, inflammatory neuropathy, toxic or drug-induced neuropathy, nutritional neuropathy, vasculitic neuropathy, paraneoplastic neuropathy, traumatic neuropathy, hereditary neuropathy and idiopathic neuropathy.

9. A method of treating neuropathy comprising administering a first component containing trimethobenzamide or a pharmaceutically acceptable salt thereof and a second component containing tramadol or a pharmaceutically acceptable salt thereof to a subject in need thereof.

10. The method of treating neuropathy according to claim 9, wherein the first component and the second component are administered sequentially or simultaneously.

11. The method of treating neuropathy according to claim 9, wherein the neuropathy is at least one neuropathy selected from the group consisting of diabetic neuropathy, postherpetic neuropathy, trigeminal neuropathy, peripheral neuropathies, peripheral mononeuropathy, mononeuritis multiplex, autonomic neuropathy, inflammatory neuropathy, toxic or drug-induced neuropathy, nutritional neuropathy, vasculitic neuropathy, paraneoplastic neuropathy, traumatic neuropathy, hereditary neuropathy and idiopathic neuropathy.
